(19) United States
(12) Patent Application Publication
(10) Pub. No.: US 2026/0087755 A1
(43) Pub. Date: Mar. 26, 2026

(54) EXTENDED REALITY GUIDANCE SYSTEM FOR VASCULAR ANATOMY REGISTRATION AND METHOD

(71) Applicant: MEDIVIEW XR, INC., Cleveland, OH (US)

(72) Inventors: Charles Martin, III, Pepper Pike, OH (US); Peter Nicholas Braido, Wyoming, MN (US); Seyedsina Shirinpour, Minneapolis, MN (US); Jeff Yanof, Solon, OH (US)

(21) Appl. No.: 19/341,327

(22) Filed: Sep. 26, 2025

Related U.S. Application Data
(60) Provisional application No. 63/699,415, filed on Sep. 26, 2024.

(51) Int. Cl. G06T 19/00; G06T 19/20; G16H 30/20; G16H 30/40

(52) U.S. Cl. CPC G06T 19/006; G06T 19/20; G16H 30/20; G16H 30/40; G06T 2219/2004

(57) ABSTRACT

A vascular access guidance platform provides enhanced visualization and accuracy for medical procedures through an integrated extended reality system. The platform comprises an extended reality visualization system configured to display three-dimensional holograms overlaid on patient anatomy, a registration and tracking system for spatial alignment between virtual and physical anatomy, multiple imaging sensors including infrared cameras and ultrasound devices, and a processing system with coordinate transformation algorithms. The system captures anatomical imaging data, generates augmented representations of vascular anatomy, and aligns these representations with physical patient anatomy using registration techniques including optical markers, spatial mapping, and bony landmark registration. The platform maintains real-time tracking capabilities throughout procedures, accommodating patient movement and practitioner repositioning. Applications include intravenous therapy, hemodialysis access, and blood sampling procedures.

### CROSS-REFERENCE TO RELATED APPLICATIONS

This application claims the benefit of U.S. Provisional Application No. 63/699,415, filed on Sep. 26, 2024. The entire disclosure of the above application is incorporated herein by reference.

### FIELD

The present technology relates to extended reality guidance systems for medical procedures, and, more particularly, to systems and methods for spatial registration and visualization of anatomical structures during vascular access and related medical interventions.

### INTRODUCTION

This section provides background information related to the present disclosure which is not necessarily prior art.

Vascular access procedures serve as fundamental components of numerous medical treatments, including hemodialysis, intravenous therapy, and blood sampling. Healthcare providers performing these procedures face persistent challenges that directly impact patient outcomes and procedural efficiency. The process of accurately locating and accessing blood vessels presents significant obstacles, particularly when treating patients with complex vascular anatomy, obesity, or other complicating anatomical factors.

Medical practitioners rely heavily on palpation techniques and visual assessment to identify suitable vascular access sites. These approaches frequently prove insufficient, leading to multiple unsuccessful insertion attempts that cause patient discomfort and increase the risk of procedural complications. The consequences of failed vascular access attempts extend beyond immediate patient discomfort, potentially resulting in bleeding, bruising, and damage to the targeted vascular access site.

Hemodialysis patients face particularly acute challenges, as their treatment regimens require vascular access procedures multiple times per week throughout their treatment course. The high frequency of these procedures compounds the risk of complications, and unsuccessful access attempts can result in missed dialysis sessions, unexpected hospital admissions, or complete abandonment of previously viable vascular access sites. These complications create cascading effects that increase healthcare costs and compromise patient care quality.

Various imaging technologies have emerged to address visualization challenges in vascular access procedures. Infrared vein finder devices represent one such technological approach, yet these systems exhibit significant limitations that prevent widespread adoption. These devices provide only two-dimensional visualization without depth information, making it difficult for healthcare providers to accurately gauge the position and depth of target blood vessels. Additionally, many of these imaging tools require handheld operation, creating interference with the practitioner's ability to efficiently perform the vascular access procedure itself.

The standard approach for creating and maintaining vascular access sites involves frequent medical interventions and imaging studies. Patients often undergo X-ray or fluoroscopy examinations every six to twelve months to visualize their vascular access circuits. Many patients require repeated procedures to maintain the functionality of their access sites. This frequent exposure to radiation and invasive procedures adds substantial burden to patient care while increasing healthcare resource utilization.

The challenges associated with vascular access procedures create a complex web of clinical, economic, and quality-of-life issues that affect both patients and healthcare providers. Failed access attempts reduce procedural efficiency, increase healthcare costs, and compromise patient comfort and safety. The limitations of existing visualization technologies leave practitioners without adequate tools to address the fundamental challenges of accurate vessel localization and depth assessment.

Accordingly, there is a continuing need for improved vascular access technologies that can enhance visualization capabilities, increase first-attempt success rates, and reduce complications associated with vascular access procedures while providing healthcare providers with hands-free operation capabilities that enable them to focus on the access procedure itself.

### SUMMARY

In concordance with the instant disclosure, improved vascular access technologies that can enhance visualization capabilities, increase first-attempt success rates, and reduce complications associated with vascular access procedures while providing healthcare providers with hands-free operation capabilities that enable them to focus on the access procedure itself have surprisingly been discovered.

In one embodiment, a vascular access guidance platform provides enhanced visualization and precision for medical procedures through an integrated extended reality system. The platform comprises an extended reality visualization system configured to display three-dimensional holograms that overlay vascular anatomy directly onto patient skin, enabling medical practitioners to visualize subcutaneous blood vessels in real-time during procedures. A registration and tracking system establishes spatial alignment between virtual anatomical representations and physical patient anatomy using sensors positioned at predetermined anatomical locations such as palpable bony landmarks at the wrist and elbow. Imaging sensors including infrared cameras, visible light cameras, and depth-sensing technology capture comprehensive anatomical information and detect vascular structures throughout the procedure. A processing system processes sensor data through coordinate transformation algorithms, generates augmented representations of patient anatomy based on captured imaging data, aligns the representations with physical anatomy using spatial registration techniques, and displays the aligned representations through the extended reality visualization system during vascular access procedures.

In another embodiment, a method for providing vascular access guidance utilizes sequential processing steps to enhance procedural accuracy and patient safety. The method begins by positioning registration sensors at predetermined anatomical locations on a patient, such as identifying bony landmarks through palpation techniques at wrist and elbow locations and placing optical sensors, electromagnetic sensors, or other suitable sensors over these anatomical reference points. Multiple imaging sensor modalities capture anatomical imaging data by utilizing infrared cameras to detect subcutaneous vascular structures, visible light cameras to capture surface anatomy, and depth-sensing technology to create three-dimensional surface maps. The method generates an augmented representation of patient anatomy at the predetermined anatomical location by processing the captured imaging data to create three-dimensional vascular maps and holographic representations suitable for vascular access guidance. Spatial registration techniques align the augmented representation with physical patient anatomy by processing sensor data from the registration sensors through coordinate transformation algorithms that establish a unified spatial reference frame. The aligned augmented representation is displayed through an extended reality visualization system that projects three-dimensional holograms overlaid directly onto patient anatomy, providing medical practitioners with enhanced visualization capabilities throughout vascular access procedures.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic diagram of the vascular access guidance platform showing the extended reality visualization system, registration and tracking system, processing system, and external imaging system interfaces.

FIG. 2 is a perspective view of the extended reality visualization system displaying holographic representations through a heads-up display during vascular access procedures.

FIG. 3 is a perspective view showing a detailed vascular anatomy hologram projected onto the patient's arm through the extended reality visualization system.

FIG. 4 is a perspective view illustrating registration using passive reflective or active LED IR fiducials positioned on anatomical landmarks with a primary fiducial and trapezoidal registration plane.

FIG. 5 is a perspective view showing registration using fiducial bands equipped with registration sensors positioned over anatomical landmarks in arc configurations.

FIG. 6 is a perspective view illustrating registration using QR codes, Vuforia 2D Image Targets, and Advanced Model Target fiducials for enhanced spatial tracking.

FIG. 7 is a perspective view showing sensorized fiducials utilizing optical and other sensor types for enhanced spatial tracking and device registration.

FIG. 8 is a perspective view showing registration using tourniquets integrated with optical and sensorized fiducials positioned on anatomical landmarks.

FIG. 9 is a perspective view showing sensorized fiducials utilizing optical and other sensor types for head and neck registration.

FIG. 10 is a perspective view showing sensorized fiducials utilizing optical and other sensor types for leg registration.

FIG. 11 is a flowchart illustrating the method with sequential steps for providing vascular access guidance.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms ‘interventional device’ or ‘tracked instrument’ refers to any medical instrument used during a medical procedure, including but not limited to needles, probes, scalpels, trocars, screwdrivers, forceps, syringes, and other surgical tools and instruments.

As used herein, the terms ‘registration device’ or ‘tracking device’ refers to any sensor or marker used to enable spatial tracking and alignment between system components, including but not limited to optical markers, electromagnetic sensors, infrared sensors, acoustic sensors, inertial measurement units, time-of-flight cameras, and other tracking devices that can observe position, orientation, or motion of system components, and combinations thereof. Registration devices may be mounted on robotic arms, treatment heads, diagnostic probes, patients, or other elements requiring spatial tracking and registration.

As used herein, the term ‘tracking system’ refers to something used to observe one or more objects undergoing motion and supply a timely ordered sequence of tracking data (e.g., location data, orientation data, or the like) in a tracking coordinate system for further processing. As an example, the tracking system can include electromagnetic tracking that can observe an interventional device equipped with a tracker, optical tracking that can detect reflective markers or light sources, fiber optic shape sensing for flexible catheters and instruments within the body, electro-anatomical mapping, impedance-based tracking, lidar or other tracking modalities that can observe the robotic or manual arm system as it moves or is positioned or interventional device as it enters, moves through and/or in, and exits a patient's body, as well as while outside of the patient's body.

As used herein, the term “tracking data” refers to information recorded by the tracking system related to an observation of one or more objects undergoing motion.

As used herein, the term “head-mounted device” or “headset” or “HMD” refers to a display device, configured to be worn on the head, that has one or more display optics (including lenses) in front of one or more eyes. These terms may be referred to even more generally by the term “augmented reality system” or “mixed reality system,” where mixed reality refers to interactive environments that combine physical objects with their related data in a manner similar to Medical Internet of Things (MIOT) applications. It should be appreciated that the term “augmented reality system” is not limited to display devices configured to be worn on the head. In some instances, the head-mounted device can also include a non-transitory memory and a processing unit. Examples of suitable head-mounted devices include Microsoft HoloLens® head-mounted device (Microsoft, Redmond, Washington), Magic Leap 2 (Magic Leap, Inc.), and DigiLens (DigiLens Inc.) devices.

The extended reality system can enable visualization through various modalities including but not limited to: augmented reality (AR) where virtual content is overlaid on the physical world, virtual reality (VR) where camera passthrough enables viewing of the physical environment within an immersive virtual space, and mixed reality (MR) where physical and virtual elements are combined in an interactive environment. The system can support multiple display types including head-mounted displays, handheld devices, and fixed position see-through displays while maintaining registration between virtual content and physical objects.

As used herein, the terms “imaging system,” “image acquisition apparatus,” “image acquisition system” or the like refer to technology that creates a visual representation of the interior of a patient's body. For example, the imaging system can be a computed tomography (CT) system, multi-detector computed tomography (MDCT), cone beam computed tomography (CBCT), photon-counting computed tomography (PCCT), a fluoroscopy system, positron emission computed tomography, magnetic resonance imaging (MRI) system, an ultrasound (US) system including contrast agents, color flow doppler, 4D ultrasound with volume rendering capabilities, or the like.

As used herein, references to specific imaging modalities such as ‘CT’ or ‘MRI’ are intended to be exemplary only. Any reference to a particular imaging modality should be understood to encompass CT, MRI, PET, and other tomographic imaging modalities that can be used interchangeably within the system for visualization, registration, and guidance purposes, unless explicitly stated otherwise.

As used herein, the terms “coordinate system” or “augmented reality system coordinate system” or “augmented reality system coordinates” refer to a 3D Cartesian coordinate system that uses one or more numbers to determine the position of points or other geometric elements unique to the particular augmented reality system or image acquisition system to which it pertains. This includes coordinate data from both internal sensors within the headset (such as inertial measurement units and optical sensors) as well as external sensors that may be attached to the headset and/or user for enhanced tracking capabilities. For example, 3D points in the headset coordinate system can be translated, rotated, scaled, or the like, from a standard 3D Cartesian coordinate system.

As used herein, the terms “image data” or “imaging dataset” or “imaging data” refers to information recorded in 3D by the imaging system related to an observation of the interior of the patient's body. For example, the “image data” or “imaging dataset” can include processed two-dimensional or three-dimensional images or models such as imaging images, e.g., represented by data formatted according to the Digital Imaging and Communications in Medicine (DICOM) standard or other relevant imaging standards.

As used herein, the terms “imaging coordinate system” or “image acquisition system coordinate system” refers to a coordinate system that uses one or more numbers to determine the position of points or other geometric elements unique to the particular imaging system. This includes three-dimensional orthogonal coordinate systems such as Cartesian coordinates, cylindrical coordinates, spherical coordinates, bipolar cylindrical coordinates, bispherical coordinates, confocal ellipsoidal coordinates, and other orthogonal curvilinear coordinate systems, as well as quaternions for representing three-dimensional rotations. For example, points and vectors in the imaging coordinate system can be translated, rotated, scaled, or the like, to the Augmented Reality system (head mounted displays) coordinate system.

As used herein, the terms “hologram”, “holographic,” “holographic projection”, or “holographic representation” refer to a computer-generated image stereoscopically projected through the lenses of a headset. Generally, a hologram can be generated synthetically (in an augmented reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two or more things being linked or coordinated. For example, a coil-sensor can be integrated with an interventional device.

As used herein, the term “real-time” or “near-real time” or “live” refers to the actual time during which a process or event occurs. In other words, a real-time event is done live (within milliseconds so that results are available immediately as feedback). For example, a real-time event can be represented within 100 milliseconds of the event occurring.

As used herein, the terms “subject” and “patient” can be used interchangeably and refer to any vertebrate organism.

As used herein, the term spatial “registration” refers to steps of transforming tracking and imaging data associated with virtual representation of tracked devices—including holographic guides, applicators, and ultrasound image stream—and additional body image data for mutual alignment and correspondence of said virtual devices and image data in the head mounted displays (world) coordinate system enabling a stereoscopic holographic projection display of images and information relative to a body of a physical patient during a procedure, for example, as further described in U.S. Patent Application Publication No. 2018/0303563 to West et al., No. 2019/10869727 to Yanof et al. and also applicant's co-owned U.S. patent application Ser. No. 17/110,991 to Black et al. and U.S. patent application Ser. No. 17/117,841 to Martin III et al., the entire disclosures of which are incorporated herein by reference. As used herein, the term ‘spatial registration techniques’ refers to methods for establishing and maintaining spatial alignment between virtual anatomical representations and physical patient anatomy, including but not limited to optical marker registration, spatial mapping using SLAM algorithms, bony landmark registration, electromagnetic tracking, and combinations thereof.

The present technology provides a vascular access guidance platform for medical procedures. The platform can be particularly well suited for vascular access applications, including intravenous therapy, hemodialysis access, and blood sampling procedures. The technology can also be applied to visualization and guidance involving other anatomical structures, including lymphatic systems, spinal anatomy, and other similar anatomical structures that share common characteristics making them suitable for augmented reality guidance including tubular or luminal structures requiring precise needle or instrument access, anatomical features with identifiable spatial relationships to palpable landmarks, and structures where real-time visualization during medical procedures would enhance accuracy and reduce complications. Other suitable anatomical applications will be apparent to those skilled in the art.

The vascular access guidance platform offers several advantages over certain vascular access technologies. The platform provides real-time, three-dimensional visualization of anatomical structures overlaid directly on the patient during procedures. The hands-free operation allows healthcare providers to focus on the access procedure itself without interference from handheld devices. The platform is configured to improve first-attempt success rates, reduce complications associated with vascular access procedures, and minimize patient discomfort. The technology also serves as an advanced training platform for medical personnel and can reduce the need for repeated interventions and radiation exposure from frequent imaging studies.

The vascular access guidance platform can include multiple integrated subsystems configured to work together for medical procedure guidance. The platform can include an extended reality visualization system, a registration and tracking system, imaging sensors, and processing capabilities configured to provide comprehensive anatomical guidance during vascular access procedures. The extended reality visualization system can be configured to display extended reality representations in the form of one or three-dimensional holograms. The extended reality representations can include two-dimensional (2D) or three-dimensional (3D) depictions of relevant information for vascular access procedures and relevant anatomical features. As examples, relevant information can include preoperative and intraoperative data, such as vascular maps, planned access trajectories, needle guidance patterns, vessel depth information, and three-dimensional vascular anatomy data.

The holographic representations can be displayed in an extended reality environment, allowing medical practitioners to view and interact with the three-dimensional vascular maps relative to the physical anatomy of the patient. The extended reality visualization system can project these holograms over the arm, leg, or other vascular access site of the patient through registration with vascular imaging sensors and the registration and tracking system. The holographic guidance can be applied across pre-procedural planning, real-time procedural guidance, and medical training phases of vascular access procedures.

The extended reality system can include a display system configured to generate these holographic representations for medical practitioners during vascular access procedures. As an example, the display system can include one or more wearable headsets with stereoscopic display capabilities, though a skilled artisan can select other suitable display technologies including tablets or projection systems. The display system can project the three-dimensional holograms directly onto patient anatomy to provide the augmented guidance.

The extended reality system can include imaging sensors configured to detect anatomical structures and registration sensors positioned on patient anatomy. As examples, the extended reality system can utilize infrared cameras to detect subcutaneous vascular structures while simultaneously detecting reflective passive markers or active LED markers placed on skin surfaces. Visible light cameras can capture surface anatomy and detect optical markers, QR codes, or image targets used for registration. The depth-sensing technology can create three-dimensional surface maps while detecting spatial mapping sensors and bony landmark positions. These imaging sensors can be in communication with the extended reality system. In some embodiments, the imaging sensors can be integrally formed into a wearable of the extended reality system. A skilled artisan can select other suitable sensor configurations based on the specific anatomical structures and clinical applications.

The vascular access guidance platform can include a registration and tracking system configured to establish and maintain spatial alignment between virtual anatomical representations and physical patient anatomy during medical procedures. The registration and tracking system can allow the augmented reality visualizations to accurately correspond to the actual anatomical structures being accessed, enabling precise guidance throughout vascular procedures. The registration and tracking system can provide real-time spatial coordination between the extended reality visualization system, imaging sensors, and patient anatomy to maintain accurate alignment as patients or practitioners move during procedures.

The registration and tracking system can incorporate multiple sensor types configured to capture spatial reference information from various sources including anatomical landmarks, surface contours, and environmental features. The registration and tracking system can establish initial registration using reference points and maintain continuous tracking throughout procedures to compensate for patient movement, practitioner repositioning, or changes in procedural setup. The registration approach can accommodate different anatomical regions and clinical scenarios while providing consistent spatial accuracy suitable for medical applications.

The registration and tracking system can include multiple sensor types configured to enable spatial alignment between virtual anatomical representations and patient anatomy. As examples, the sensor types can include optical sensors configured to detect reflective passive markers or active LED markers, electromagnetic sensors for wireless tracking, acoustic sensors for position detection, and inertial measurement unit sensors for motion tracking. The sensors can be positioned on anatomical landmarks, integrated within fiducial bands, or distributed across various locations as selected by a skilled artisan. The sensor systems can work individually or in combination to provide comprehensive spatial registration data throughout medical procedures. Fiducial bands can refers to flexible wearable structures configured to be positioned around or over anatomical regions. The bands can include one or more registration targets or sensors arranged in arc configurations that conform to the cross-sectional geometry of target anatomical regions such as forearms, upper arms, or legs. The fiducial bands are configured to maintain continuous spatial registration by providing stable reference points while accommodating anatomical contours and patient movement during medical procedures.

Optical sensors can capture light reflection patterns from reflective passive markers or light emission signals from active LED markers positioned on anatomical landmarks. The sensors can process the captured optical data through triangulation algorithms that calculate three-dimensional coordinates by measuring the angular relationships and distances between multiple detected marker positions. The optical processing can include real-time image analysis algorithms that identify marker centroid positions, filter background noise, and convert pixel coordinates into world coordinate spatial positions relative to the extended reality system coordinate frame.

Electromagnetic sensors can generate and detect electromagnetic field signatures to determine spatial positions through field strength analysis and signal timing measurements. The electromagnetic processing can utilize field mapping algorithms that convert electromagnetic signal characteristics into six-degree-of-freedom positional data including translation and rotation coordinates. The sensor system can process electromagnetic field distortions and signal propagation delays to calculate precise spatial coordinates even when optical line-of-sight is obstructed.

Spatial mapping sensors using SLAM algorithms can process environmental scanning data to create comprehensive three-dimensional coordinate maps of patient anatomy and surrounding surfaces. The processing can include real-time point cloud generation, surface reconstruction algorithms, and feature detection methods that convert depth sensor data into navigable coordinate systems. The spatial mapping processing can continuously update coordinate transformations as new spatial information is captured, maintaining accurate registration throughout medical procedures.

IMU sensors can process accelerometer, gyroscope, and magnetometer data through sensor fusion algorithms that calculate position, orientation, and motion parameters in real-time. The IMU processing can include drift compensation algorithms, noise filtering, and coordinate transformation matrices that convert raw sensor measurements into stable spatial coordinate data. The sensor fusion processing can combine multiple IMU data streams to enhance positional accuracy and provide continuous motion tracking capabilities.

Time-of-flight sensors can measure distance information by calculating the time required for emitted light signals to return after reflecting from anatomical surfaces. The processing can include signal timing analysis, distance calculation algorithms, and coordinate mapping functions that convert time-of-flight measurements into precise three-dimensional surface coordinates. The depth processing can generate comprehensive surface coordinate maps that enable accurate registration of virtual anatomical representations to physical patient anatomy.

The registration and tracking system can process data from multiple sensor types simultaneously through coordinate transformation algorithms that align different sensor coordinate systems into a unified spatial reference frame. The integration processing can include coordinate system calibration, temporal synchronization, and sensor fusion algorithms that combine optical, electromagnetic, spatial mapping, and inertial data into coherent spatial coordinate information. The multi-modal processing can provide enhanced accuracy and reliability compared to individual sensor approaches while maintaining real-time performance throughout vascular access procedures.

The system can utilize individual sensors, combinations of multiple sensor types, or other suitable sensor configurations as selected by a skilled artisan based on the specific procedure to be performed. As examples, for basic intravenous access procedures, the system can operate using a single infrared camera sensor to detect subcutaneous vascular structures, while more complex fistula access procedures can utilize combinations of infrared cameras, depth-sensing technology, and electromagnetic sensors working together to provide enhanced spatial registration accuracy. For head and neck applications, the system can incorporate ultrasound sensors as the primary imaging modality combined with facial recognition sensors for registration, while leg procedures can utilize infrared imaging sensors combined with spatial mapping sensors for comprehensive vascular visualization. The system architecture can accommodate sensor configurations not specifically enumerated herein, enabling a skilled artisan to select optimal sensor combinations based on anatomical region, patient characteristics, clinical requirements, or procedural complexity while maintaining the core functionality of spatial registration and augmented reality guidance. The modular sensor approach can enable the system to adapt to evolving medical procedures and emerging sensor technologies without requiring fundamental architectural modifications.

The sensors can be positioned at pre-selected locations related to specific anatomical structures to optimize registration accuracy and spatial alignment. As examples, sensors can be placed over palpable bony landmarks that serve as stable anatomical reference points, positioned along anatomical segments where consistent spatial relationships exist, or arranged in geometric configurations that correspond to anatomical geometry patterns. The sensor positioning can accommodate different anatomical regions including arm and forearm locations utilizing wrist and elbow landmarks, leg locations utilizing ankle and knee landmarks, and head and neck locations utilizing facial and skeletal reference points. A skilled artisan can select other suitable anatomical locations and sensor positioning strategies based on the target anatomical region and procedural requirements. For arm registration applications, sensors can be positioned at specific bony landmarks identified through palpation techniques at the wrist and elbow locations. As examples, wrist sensor placement can utilize the dorsal tubercle of radius, styloid process of ulna, and styloid process of radius as reference points, while elbow sensor placement can utilize the lateral supracondylar ridge, lateral epicondyle, and radial fossa as anatomical landmarks. By identifying and sensing specific bony landmarks at consistent anatomical locations, the system can establish spatial reference frameworks that correspond to predictable anatomical geometry patterns. As examples, three sensors placed on bony landmarks can form triangular registration planes that establish spatial coordinates for the anatomical region, while four or more sensors can create trapezoidal planes that provide enhanced spatial resolution and area coverage. These geometric configurations can enable the system to map the anatomical area between the landmark points by utilizing the known spatial relationships and anatomical connections that exist within the geometric boundaries.

The extended reality system can process and combine multiple imaging input sources to create comprehensive augmented reality visualizations for vascular access procedures. The system can integrate real-time sensor data captured during procedures with pre-procedural imaging data obtained from existing medical imaging systems, and intraprocedural ultrasound data acquired during active medical interventions. The processing system can utilize image fusion algorithms that combine these diverse imaging inputs into coherent three-dimensional anatomical representations suitable for holographic display through the extended reality visualization system.

Real-time sensor processing can capture anatomical information through the imaging sensor array during active procedures. As examples, infrared cameras can detect subcutaneous vascular structures in real-time by processing differential light absorption patterns, while visible light cameras can capture surface anatomy and environmental mapping data. Depth-sensing technology can generate three-dimensional surface coordinate data through time-of-flight processing algorithms, and the system can process this real-time sensor data through image enhancement algorithms that improve vessel visibility and differentiate between anatomical structures. A skilled artisan can select other suitable real-time processing approaches based on the specific sensor configurations and clinical requirements.

Pre-procedural imaging data integration can incorporate existing medical imaging information into the augmented reality display to enhance procedural guidance. T “The system can interface with fluoroscopy systems, X-ray systems, CT imaging, MRI systems, ultrasound systems, or other medical imaging modalities to receive and process pre-acquired anatomical data. As examples, for fistula procedures, the system can integrate X-ray and fluoroscopy imaging performed pre-procedurally to map vascular circuits with enhanced depth information and precise anatomical localization. The processing system can align pre-procedural imaging data with real-time sensor inputs through coordinate transformation algorithms that register different imaging coordinate systems into the extended reality system reference frame.

Intraprocedural ultrasound integration can provide real-time high-resolution imaging of deeper anatomical structures during active medical procedures. The system can process ultrasound data streams from miniaturized ultrasound sensors integrated within the extended reality system or from external ultrasound devices that interface with the platform. As examples, for head and neck applications, ultrasound can serve as the primary imaging modality providing enhanced soft tissue visualization, while for complex vascular access procedures, intraprocedural ultrasound can supplement infrared imaging with deeper vessel detection capabilities. The ultrasound processing can include real-time image enhancement, tissue differentiation algorithms, and spatial coordinate mapping that aligns ultrasound data with other sensor inputs.

Multi-modal image fusion processing can combine data from real-time sensors, pre-procedural imaging systems, and intraprocedural ultrasound through sophisticated algorithms that create unified anatomical representations. The fusion processing can include temporal synchronization algorithms that align imaging data captured at different time points, spatial registration algorithms that coordinate different imaging coordinate systems, and image enhancement algorithms that optimize the combined data for holographic display. As examples, the fusion processing can combine real-time infrared vascular detection with pre-procedural fluoroscopy circuit mapping and intraprocedural ultrasound depth information to create comprehensive three-dimensional vascular maps. The system can weight different imaging inputs based on data quality, temporal relevance, and spatial accuracy to optimize the final augmented representation.

The combined imaging data processing can generate augmented representations that incorporate the enhanced anatomical information from all imaging sources into the holographic displays. The processing system can create layered visualizations where pre-procedural imaging data provides baseline anatomical mapping, real-time sensor data provides current procedural context, and intraprocedural ultrasound data provides enhanced depth and tissue differentiation. The augmented representations can dynamically adjust based on the availability and quality of different imaging inputs, enabling the system to maintain effective guidance even when individual imaging modalities experience limitations or interruptions. A skilled artisan can configure the image fusion parameters and processing priorities based on the specific procedural requirements and available imaging resources.

The system can incorporate physiological and dynamic visualization capabilities that provide real-time monitoring of vascular flow patterns and hemodynamic changes during procedures. Doppler ultrasound integration can enable visualization of blood flow velocities, flow directions, and flow characteristics overlaid as dynamic holographic representations within the extended reality environment. The processing system can process Doppler ultrasound data streams to generate real-time flow visualization maps that display velocity vectors, turbulence patterns, and flow timing information directly onto patient anatomy through the extended reality visualization system.

The infrared imaging capabilities can be enhanced with flow and pressure detection algorithms that monitor physiological changes in real-time during vascular access procedures. The system can detect infrared signature variations corresponding to blood flow changes, pressure fluctuations, and vascular filling patterns to provide dynamic feedback about optimal timing and positioning for device application. The processing system can correlate infrared flow detection with pressure mapping algorithms to generate comprehensive physiological guidance that indicates when and where to apply vascular access devices based on real-time hemodynamic conditions.

The registration and tracking system can interface with the extended reality visualization system through multiple data communication pathways configured to maintain real-time spatial alignment throughout vascular access procedures. The registration system can transmit spatial coordinate data, orientation information, and tracking updates to the visualization system through wireless communication protocols or direct data connections. As examples, the interface can utilize high-speed data transmission protocols configured to support real-time registration updates, though a skilled artisan can select other suitable communication methods based on the specific system configuration and clinical requirements. The data flow interface can enable the registration and tracking system to provide continuous spatial registration information to the extended reality visualization system for holographic alignment adjustments. The registration system can transmit tracking data including sensor positions, anatomical landmark coordinates, and spatial mapping information that the visualization system processes to maintain accurate overlay positioning. As examples, the data flow can include real-time coordinate transformations, registration quality metrics, and motion compensation data, though a skilled artisan can select other suitable data transmission formats based on the procedural requirements.

The interface system can coordinate processing activities between the registration and tracking system and the extended reality visualization system to synchronize spatial alignment with holographic display updates. The registration system can provide timing signals and synchronization data that enable the visualization system to coordinate holographic projection updates with registration tracking cycles. The interface can incorporate feedback mechanisms where the visualization system communicates display status and rendering information back to the registration system for optimized spatial alignment accuracy.

The user interface integration can enable the registration and tracking system to communicate system status and calibration information to medical practitioners through the extended reality visualization display. As examples, the interface can display registration quality indicators, sensor connectivity status, and calibration prompts within the augmented reality environment, though a skilled artisan can select other suitable user interface elements based on the clinical workflow requirements. The registration system can provide error notifications, accuracy assessments, and system diagnostic information that the visualization system presents to practitioners during vascular access procedures.

The interface architecture can enable bi-directional communication where the extended reality visualization system provides user input and interaction data back to the registration and tracking system for system optimization. The visualization system can transmit user gesture data, voice commands, and interaction preferences to the registration system for adaptive tracking behavior and personalized registration approaches. The interface can support dynamic system configuration adjustments where user preferences and procedural requirements modify registration tracking parameters and sensor fusion algorithms in real-time.

The vascular access guidance platform can integrate into existing clinical workflows across multiple phases of medical procedures. The integration can accommodate pre-procedural planning phases where medical practitioners review patient anatomy and plan access strategies, intra-procedural guidance phases where real-time visualization supports active medical interventions, and post-procedural documentation phases where system data can be recorded for quality assessment and training purposes. As examples, the workflow integration can support routine intravenous therapy procedures, complex hemodialysis access procedures, and specialized vascular interventions, though a skilled artisan can adapt the system for other suitable clinical applications based on procedural requirements.

The system can provide workflow support for different categories of healthcare providers including nurses, technicians, physicians, and trainees performing vascular access procedures. The workflow integration can adapt to different skill levels and procedural complexity requirements through configurable interface options and guidance modes. As examples, the system can provide basic guidance for routine IV access procedures or advanced guidance for complex fistula procedures requiring enhanced spatial precision. The platform can support training workflows where novice practitioners can learn proper techniques through augmented reality guidance while experienced practitioners can utilize the system for challenging cases.

The clinical workflow integration can include quality monitoring and documentation capabilities that support procedural assessment and continuous improvement initiatives. The system can record procedural data including access attempt success rates, procedure timing, and spatial accuracy metrics that can be reviewed for quality assessment purposes. As examples, the workflow integration can generate reports for healthcare administrators, provide feedback for practitioner training programs, or contribute data for clinical research studies, though a skilled artisan can select other suitable documentation approaches based on institutional requirements. The integration can support both individual practitioner performance assessment and broader healthcare system quality improvement initiatives.

The method can include sequential processing steps configured to provide comprehensive anatomical guidance during medical procedures. The method can accommodate different anatomical regions and clinical scenarios while maintaining consistent spatial accuracy suitable for vascular access applications. As examples, the method can be applied to intravenous therapy procedures, hemodialysis access procedures, and blood sampling procedures, though a skilled artisan can adapt the method for other suitable medical applications based on procedural requirements.

The imaging step can utilize multiple sensor modalities working individually or in combination to capture comprehensive anatomical information. As examples, the imaging can include infrared cameras detecting subcutaneous vascular structures, visible light cameras capturing surface anatomy, depth-sensing technology creating three-dimensional surface maps, and ultrasound sensors providing deeper tissue visualization. The imaging can be performed both pre-procedurally and during medical procedures, utilizing integral sensors of extended reality systems or external imaging systems that interface with the display system. A skilled artisan can select other suitable imaging approaches based on the specific anatomical region, vessel depth requirements, and procedural timing considerations.

The generating step can process the anatomical imaging data through algorithms configured to enhance anatomical visualization and construct three-dimensional representations. The generation process can enhance vessel visibility, differentiate between different anatomical structures, and create coherent spatial maps from various imaging inputs. As examples, the generating can include image processing algorithms for vessel enhancement, machine learning algorithms for anatomical recognition, and real-time reconstruction algorithms for spatial mapping. The generating step can incorporate pre-procedural imaging data from existing medical imaging modalities such as X-ray or fluoroscopy systems to enhance the augmented representation.

The aligning step can establish and maintain spatial correspondence between the generated augmented representation and the physical anatomy of the patient. The alignment process can utilize various registration techniques including optical marker registration, spatial mapping algorithms, bony landmark registration, or combinations of multiple registration approaches. As examples, the aligning can include processing data from sensors positioned at predetermined anatomical locations, calculating coordinate transformations between different spatial reference systems, and maintaining real-time registration updates throughout procedures. The aligning step can accommodate patient movement and practitioner repositioning through dynamic registration techniques that provide continuous spatial alignment.

The displaying step can present the aligned augmented representation through various display technologies configured to provide real-time guidance during medical procedures. As examples, the displaying can include presenting three-dimensional holograms through wearable headsets, projecting augmented representations through AR projectors, or displaying anatomical information through tablets or screens. The displaying step can overlay the anatomical information directly onto the patient during procedures, enabling medical practitioners to visualize target anatomy while performing vascular access procedures. The displaying can provide applications across pre-procedural planning, real-time procedural guidance, and medical training phases of vascular access procedures.

### EXAMPLES

Example embodiments of the present technology are provided with reference to the several figures enclosed herewith. The following examples describe specific embodiments and implementations of the vascular access guidance platform for illustrative purposes and are not intended to limit the scope of the present technology. The examples demonstrate particular configurations of the extended reality visualization system, registration and tracking system, and imaging sensor arrays for various anatomical applications. A skilled artisan can adapt these examples and select other suitable configurations, sensor types, registration techniques, or anatomical applications based on specific procedural requirements and clinical considerations. The examples focus primarily on arm registration and vascular access applications, though the technology can be readily applied to other anatomical regions including leg procedures, head and neck applications, and other suitable anatomical structures where augmented reality guidance would provide clinical benefits. The registration techniques, sensor configurations, and processing approaches described in these examples can be modified, combined, or substituted with alternative implementations while maintaining the core functionality of spatial registration and augmented reality visualization. Other like structures that share similar characteristics with vascular anatomy, including lymphatic systems, spinal canals, and tubular or luminal structures requiring precise instrument access, can be visualized using the same fundamental principles and adapted system configurations.

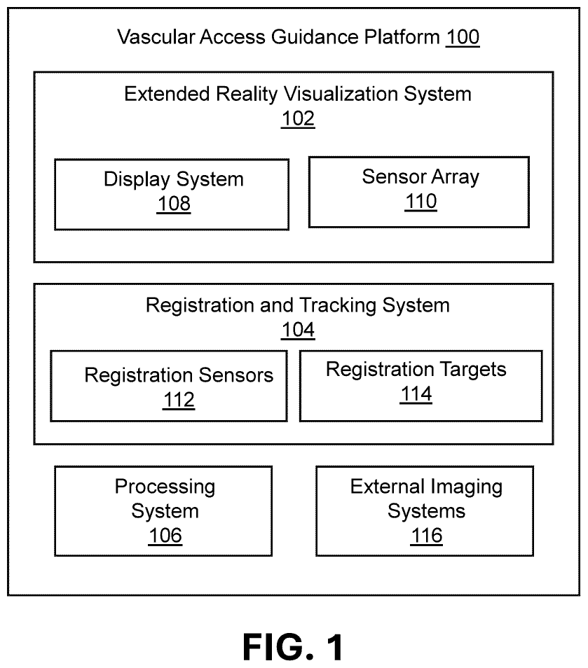

FIG. 1 illustrates a schematic diagram of the vascular access guidance platform 100 configured for arm registration applications. The platform 100 can include an extended reality visualization system 102, registration and tracking system 104, processing system 106, and external imaging system interfaces 116 working together to provide comprehensive anatomical guidance during vascular access procedures. The extended reality visualization system 102 can include display system components 108 configured to present three-dimensional holographic representations overlaid on patient anatomy. The imaging sensor array 110 can incorporate multiple sensor modalities including infrared cameras for detecting subcutaneous vascular structures and visible light cameras for surface mapping.

The registration and tracking system 104 can utilize registration targets 114 positioned at predetermined anatomical locations on the patient's arm. As shown, registration targets 114 can be placed over specific bony landmarks identified through palpation techniques. For arm registration, these anatomical reference points can include wrist landmarks such as the dorsal tubercle of radius, styloid process of ulna, and styloid process of radius, as well as elbow landmarks including the lateral supracondylar ridge, lateral epicondyle, and radial fossa. The registration sensors 112 can include optical sensors configured to detect reflective passive markers or active LED markers, electromagnetic sensors for wireless tracking, and inertial measurement unit sensors positioned on the identified anatomical landmarks. The registration targets 114 can be arranged in geometric configurations that form triangular or trapezoidal registration planes to establish spatial coordinates for the anatomical region. The system 100 can allow for a visualization of an access area 103 on the patient 101.

The external imaging system interfaces 116 can enable integration with existing medical imaging modalities to enhance vascular anatomy visualization during procedures. As examples, the external imaging interfaces can connect with fluoroscopy systems for pre-procedural vascular circuit mapping, X-ray systems for enhanced depth information, ultrasound devices for real-time deeper vessel imaging, or other medical imaging systems through standard communication protocols. The external imaging system interfaces 116 can process and integrate pre-procedural imaging data with real-time sensor data to provide comprehensive vascular mapping capabilities. For fistula procedures, the external imaging interfaces can incorporate fluoroscopy and X-ray data obtained pre-procedurally to map vascular circuits with enhanced spatial accuracy.

The processing system 106 can process sensor data from both the integrated imaging sensor array 110 and external imaging system interfaces 116 through coordinate transformation algorithms that align the registration sensor inputs into a unified spatial reference frame, enabling the extended reality visualization system 102 to accurately overlay vascular anatomy visualizations on the patient's physical arm during vascular access procedures. The system can maintain accurate spatial alignment as patients or practitioners move during procedures through real-time tracking capabilities and continuous registration updates, while incorporating both real-time and pre-procedural imaging data for enhanced procedural guidance.

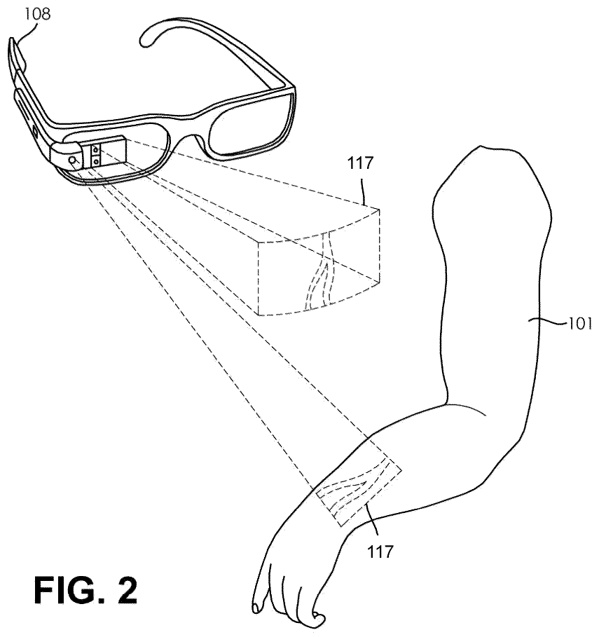

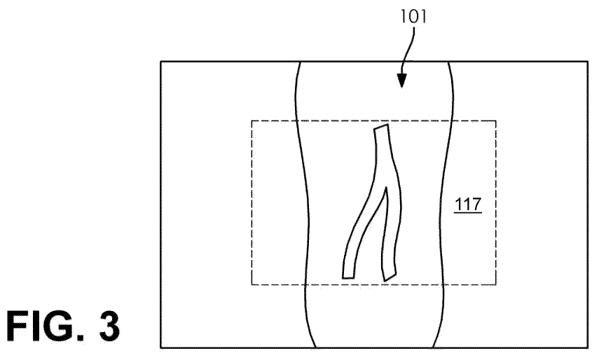

FIG. 2 illustrates the extended reality visualization system 102 configured to display holographic representations through a heads-up display during vascular access procedures. The display system components 108 can present three-dimensional holograms 117 101 overlaid directly on patient anatomy, showing the arm, headset configuration, and projections of vascular anatomy in real-time. The wearable headset can include imaging sensor array 110 configured to capture anatomical information while simultaneously displaying the augmented vascular representations through the heads-up display interface. The holographic projections can include vascular maps, vessel depth information, and three-dimensional vascular anatomy data that align with the physical anatomy through the registration and tracking system 104. The display system can present the vascular anatomy projections as stereoscopic holograms that medical practitioners can view relative to the patient's physical arm during procedures. FIG. 3 presents a detailed view of the vascular anatomy hologram 117 projected onto the patient's arm through the extended reality visualization system 102.

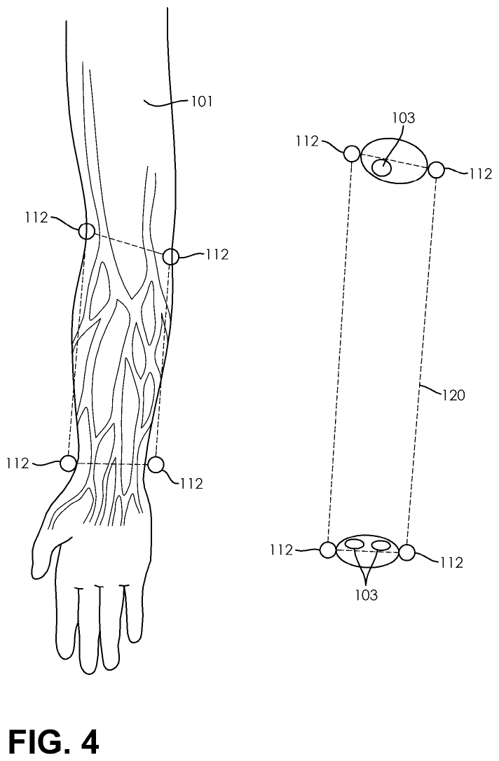

FIG. 4 illustrates a registration method for arm vascular access utilizing passive reflective or active LED IR fiducials positioned on anatomical landmarks. The registration and tracking system 104 can utilize registration targets 114 placed over bony protrusions identified through palpation techniques at the wrist and elbow locations. The registration sensors 112 can include optical sensors configured to detect reflective passive markers or active LED markers positioned directly on the identified anatomical landmarks. As shown, a primary fiducial 118 can be positioned on a specific bony landmark while additional markers form a trapezoidal registration plane 120 that establishes spatial coordinates for the anatomical region.

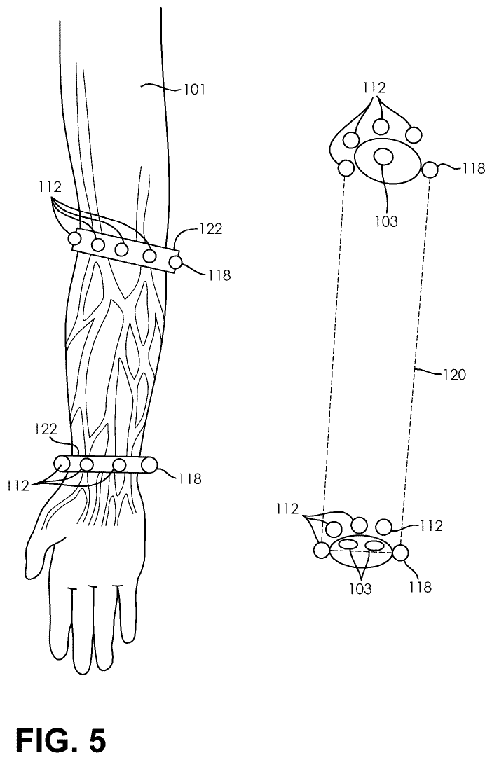

FIG. 5 illustrates an alternative registration approach utilizing fiducial bands 122 equipped with registration targets 114 positioned over anatomical landmarks for enhanced spatial tracking during vascular access procedures. The registration and tracking system 104 can incorporate sensor bands that can be placed over bony protrusions identified through palpation techniques, providing consistent reference points while accommodating the cross-sectional geometry of the anatomical region. The fiducial bands can include multiple registration targets 114 arranged in arc configurations that represent the cross-section of the forearm or other target anatomical region. As shown, the primary fiducial 118 can be positioned to align with a specific bony landmark while additional markers on the band form an arc pattern that corresponds to the anatomical contours. This arrangement can enable the registration sensors 112 to maintain continuous spatial registration as the bands conform to the patient anatomy.

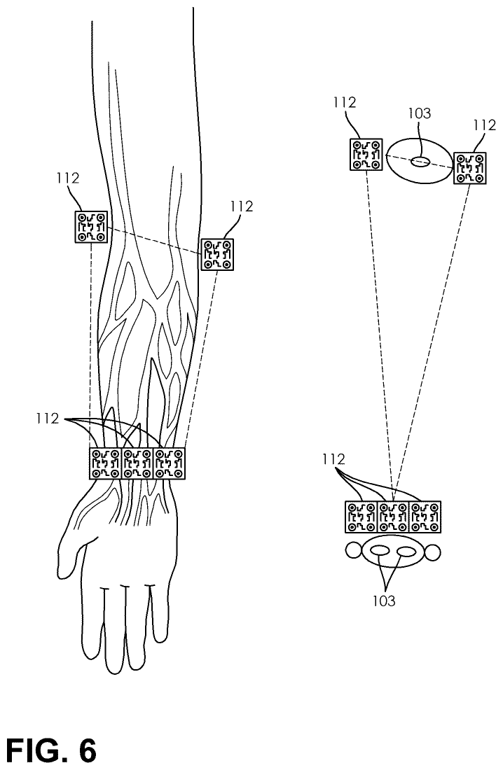

FIG. 6 illustrates an advanced registration approach utilizing image target registration with QR codes, Vuforia 2D Image Targets, and Advanced Model Target (AMT) fiducials for enhanced spatial tracking during vascular access procedures. The registration and tracking system 104 can incorporate image-based registration sensors 112 that can be adhered to skin surfaces over anatomical landmarks to provide additional registration reference points. The registration targets 114 can include (e.g., QR codes, Vuforia 2D Image Targets, optical codes or the like) positioned directly over bony protrusions identified through palpation techniques. As examples, the image targets can be placed over wrist and elbow landmarks to form triangular or polygonal registration planes 120 that establish spatial coordinates for the anatomical region. The image target fiducials can lay on specific bony landmarks while additional markers form geometric configurations that enable the registration and tracking system 104 to register vascular image data in three-dimensional real-time space.

For enhanced tracking precision, the registration targets 114 can be designed as unique three-dimensional objects that provide six degrees of freedom (6DOF) information to the extended reality visualization system 102. As examples, asymmetric polyhedral objects can be placed at each anatomical landmark, with textured surfaces and different colors on each side to add comprehensive image data for tracking. The system can also incorporate RFID stickers with integrated readers adhered to skin surfaces over landmarks for wireless identification and positioning. The imaging sensor array 110 can detect and process the image target patterns through computer vision algorithms that identify unique visual features and calculate precise spatial positioning relative to the extended reality system coordinate frame. The image-based registration approach can provide robust tracking capabilities that maintain accurate spatial alignment throughout vascular access procedures while accommodating various lighting conditions and procedural environments.

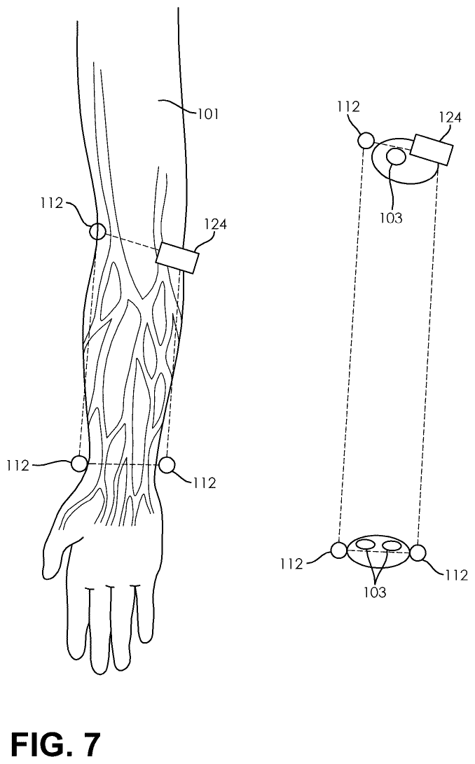

FIG. 7 illustrates an advanced sensorized fiducial approach utilizing optical and other sensor types for enhanced spatial tracking and device registration during vascular access procedures. The registration and tracking system 104 can incorporate multi-modal sensor fiducials that combine optical tracking with additional sensing modalities to improve positional accuracy and enable registration of medical instruments within the holographic space. The registration sensors 112 can include optical fiducial sensors 124 equipped with acoustic sensors, electromagnetic sensors, or inertial measurement unit sensors positioned on anatomical landmarks. This multi-sensor approach can enhance the positional accuracy of the registration targets 114 while providing additional sensing capabilities that complement the optical tracking system. As examples, the sensorized fiducials can incorporate wireless electromagnetic tracking, acoustic position detection, or motion sensing through IMU sensors as selected by a skilled artisan based on procedural requirements.

The enhanced sensor configuration can enable registration of additional medical devices within the extended reality visualization system 102. As shown, vascular access instruments such as needles can be equipped with one or more of the optical sensors, electromagnetic sensors, acoustic sensors, or IMU sensors to register the device position relative to the patient anatomy in three-dimensional holographic space. The processing system 106 can process data from the multiple sensor types simultaneously to maintain accurate spatial alignment between the registered medical instruments, registration targets 114, and the vascular anatomy throughout the procedure.

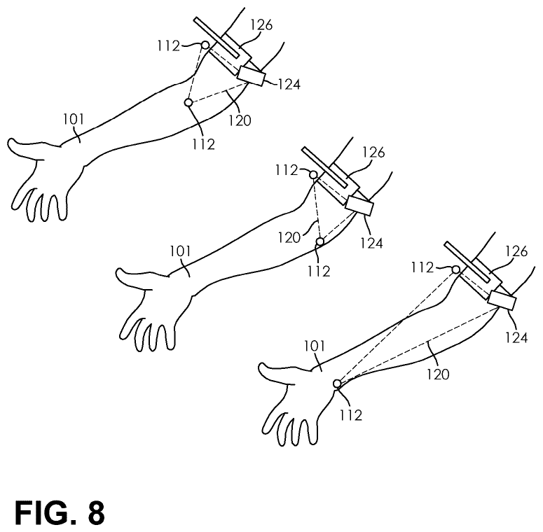

FIG. 8 illustrates an alternative registration approach for arm vascular access utilizing tourniquets 126 integrated with optical and other sensorized fiducials positioned on anatomical landmarks. The registration and tracking system 104 can incorporate tourniquets as functional components that serve dual purposes as medical devices and registration reference platforms during vascular access procedures. The torniquet 126 registration approach can utilize a flexible band, such as a tourniquet, compression band, or other wearable band structure, as a carrier for two or more registration sensors 112 configured to enable image fusion of known skin surface data with bony landmark information.

As shown, the registration sensors 112 can be positioned on palpated bony landmarks at the elbow and wrist locations while additional fiducials can be placed on skin surfaces to establish comprehensive spatial registration. The band can accommodate various shapes, designs, and materials suitable for different anatomical locations while providing stable platforms for sensor positioning and maintaining consistent contact with the skin surface. The registration configuration can utilize triangular registration planes formed by three registration sensors 112 positioned at strategic anatomical locations, as opposed to the trapezoidal configurations described in other embodiments. The triangular arrangement can provide effective spatial coordinate establishment while accommodating the constraints imposed by band positioning and clinical workflow requirements. The band can include one of the sensorized fiducials 124 that can include optical sensors, electromagnetic sensors, acoustic sensors, or inertial measurement unit sensors integrated within or mounted on the band structure.

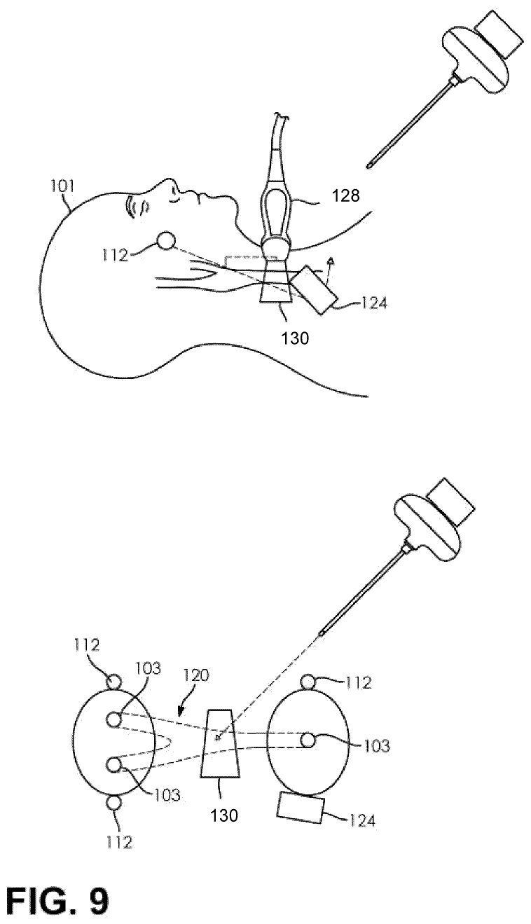

FIG. 9 illustrates a registration approach for head and neck vascular access utilizing ultrasound as the primary imaging modality for enhanced soft tissue and vascular structure visualization. The registration and tracking system 104 can accommodate the complex anatomical structures in the head and neck region by incorporating ultrasound sensors within the imaging sensor array 110 and external imaging system interfaces 116. The registration sensors 112 can utilize bony landmarks such as the zygomatic arch and sternoclavicular joint as anatomical reference points for spatial alignment. As shown, the registration targets 114 can be positioned over these facial and skeletal landmarks to establish spatial coordinates for the head and neck anatomical region.

The extended reality visualization system 102 can incorporate facial recognition technology, with or without optical sensors, to enhance the registration process by leveraging unique facial features of each patient to improve accuracy of the AR overlay alignment. The ultrasound wand 128 can be equipped with registration sensors 112 and can interface with external imaging system interfaces 116 to enable the processing system 106 to track the ultrasound probe position and orientation within the three-dimensional holographic space. The external imaging system interfaces 116 can process ultrasound data streams from the sensorized ultrasound device while maintaining spatial registration with the extended reality visualization system 102 throughout head and neck vascular access procedures. The system 100 can likewise project the ultrasound plane 130 onto the anatomy of the patient.

The system 100 can also accommodate registration of lymphatic structures through palpation of lymph nodes to register lymphatic lumens, expanding the anatomical applications beyond traditional vascular access procedures. The head and neck registration configuration can maintain the same multi-modal registration capabilities while adapting the primary imaging modality to ultrasound through the external imaging system interfaces 116 for optimal visualization in this anatomical region.

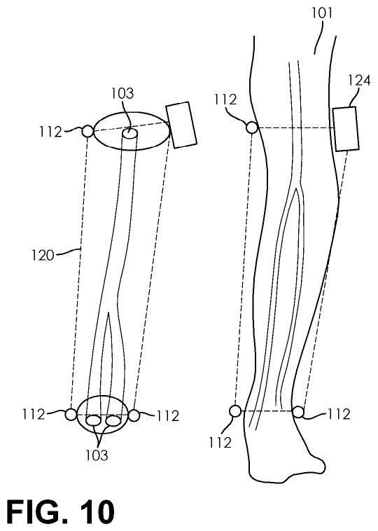

FIG. 10 illustrates a registration approach for leg vascular access utilizing sensorized fiducials positioned on anatomical landmarks for enhanced spatial tracking during lower extremity vascular procedures. The registration and tracking system 104 can accommodate the complex anatomical structures in the leg region by incorporating multiple sensor modalities within the imaging sensor array 110 to provide comprehensive visualization of deep and superficial vascular structures including the popliteal artery, anterior tibial artery, posterior tibial artery, and fibular artery.

The registration sensors 112 can utilize bony landmarks such as the medial and lateral malleoli at the ankle and the tibial condyles at the knee as anatomical reference points for spatial alignment. As shown, the registration targets 114 can be positioned over these palpable bony landmarks to establish spatial coordinates for the leg anatomical region. The extended reality visualization system 102 can incorporate infrared imaging capabilities to visualize the femoral vein relative to the bony structures, providing enhanced visualization of deep vascular anatomy that is particularly beneficial for leg vascular access procedures.

The sensorized fiducials can be equipped with registration sensors 112 and can interface with the processing system 106 to enable comprehensive spatial tracking throughout leg vascular access procedures. Similar to the arm registration applications, the leg registration approach can utilize optical sensors configured to detect reflective passive markers or active LED markers, electromagnetic sensors for wireless tracking, acoustic sensors for position detection, and inertial measurement unit sensors positioned on the identified anatomical landmarks. The registration targets 114 can be arranged in geometric configurations that form triangular or trapezoidal registration planes adapted to the longer anatomical segments of the leg region.

The processing system 106 can process sensor data from the registration targets 114 positioned at ankle and knee locations to maintain accurate spatial alignment between the extended reality visualization system 102 and the patient's physical leg anatomy during vascular access procedures. The leg registration configuration can accommodate the enhanced visualization requirements for lower extremity procedures where deeper tissue imaging and visualization of arterial structures can be particularly beneficial for accurate vascular access. The system can maintain the same multi-modal registration capabilities while adapting the sensor positioning and geometric configurations to optimize spatial coverage across the longer anatomical distances typical of leg applications compared to arm procedures.

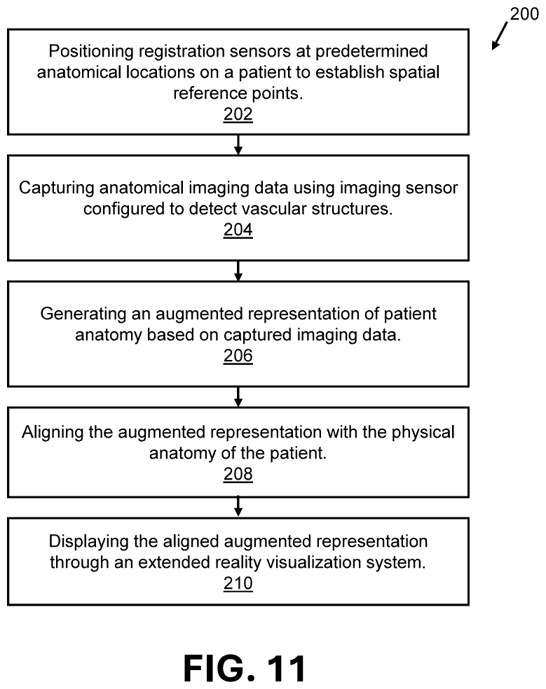

As shown in FIG. 11, the method 200 can include a step 202 of positioning registration sensors at predetermined anatomical locations on a patient to establish spatial reference points for augmented reality guidance during vascular access procedures. Step 202 can include identifying bony landmarks through palpation techniques at wrist and elbow locations for arm procedures, or ankle and knee locations for leg procedures. The step can include placing optical sensors, electromagnetic sensors, acoustic sensors, or inertial measurement unit sensors over the identified anatomical landmarks as selected by a skilled artisan based on procedural requirements.

The method 200 can include a step 204 of capturing anatomical imaging data using multiple imaging sensor modalities configured to detect vascular structures and anatomical features. Step 204 can include utilizing infrared cameras to detect subcutaneous vascular structures, visible light cameras to capture surface anatomy, depth-sensing technology to create three-dimensional surface maps, and ultrasound sensors to provide deeper tissue visualization.

The step can include processing the imaging data through algorithms that enhance vessel visibility and differentiate between anatomical structures.

The method 200 can include a step 206 of generating an augmented representation of patient anatomy at the predetermined anatomical location based on the captured imaging data. Step 206 can include processing the anatomical imaging data to create three-dimensional vascular maps, holographic representations, and spatial visualizations suitable for vascular access guidance. The step can include incorporating pre-procedural imaging data from external medical imaging systems such as fluoroscopy or X-ray systems to enhance the augmented representation.

The method 200 can include a step 208 of aligning the augmented representation with the physical anatomy of the patient using spatial registration techniques. Step 208 can include processing sensor data from the registration sensors through coordinate transformation algorithms that establish a unified spatial reference frame. The step can include utilizing optical marker registration, spatial mapping using SLAM algorithms, bony landmark registration, or combinations of multiple registration approaches to maintain accurate spatial alignment throughout the procedure.

The method 200 can include a step 210 of displaying the aligned augmented representation through an extended reality visualization system during vascular access procedures. Step 210 can include projecting three-dimensional holograms overlaid directly onto patient anatomy through wearable headsets or other suitable display technologies. The step can include maintaining real-time tracking capabilities that provide continuous registration updates as patients or practitioners move during procedures.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A vascular access guidance platform for medical procedures on a patient, comprising:
an extended reality visualization system configured to display extended reality representations in the form of three-dimensional holograms of anatomy of the patient;
a registration and tracking system configured to establish and maintain spatial alignment between virtual anatomical representations and physical patient anatomy during medical procedures;
imaging sensors configured to capture anatomical information and detect anatomical structures, the imaging sensors in communication with the extended reality visualization system; and
a processing system configured to:
process sensor data from the imaging sensors,
generate an augmented representation of patient anatomy based on captured imaging data,
align the augmented representation with physical anatomy of the patient, and
display the aligned augmented representation through the extended reality visualization system during vascular access procedures.

2. The vascular access guidance platform of claim 1, wherein the extended reality visualization system includes a display system comprising a wearable headset with stereoscopic display capabilities configured to project the three-dimensional hologram directly onto patient anatomy.

3. The vascular access guidance platform of claim 1, wherein the imaging sensors include one of an infrared camera configured to detect subcutaneous vascular structures, a visible light camera configured to capture surface anatomy, and depth-sensing technology configured to create three-dimensional surface maps.

4. The vascular access guidance platform of claim 1, wherein the registration and tracking system includes a registration sensor selected from the group consisting of an optical sensor, an electromagnetic sensor, an acoustic sensor, an inertial measurement unit sensor, and combinations thereof.

5. The vascular access guidance platform of claim 1, wherein the registration and tracking system is configured to position sensors at predetermined anatomical locations.

6. The vascular access guidance platform of claim 5, wherein for arm registration applications, the predetermined anatomical locations include wrist landmarks include a dorsal tubercle of radius, styloid process of ulna, and styloid process of radius, and elbow landmarks comprising a lateral supracondylar ridge, lateral epicondyle, and radial fossa.

7. The vascular access guidance platform of claim 1, wherein the processing system is configured to integrate real-time sensor data captured during procedures with pre-procedural imaging data obtained from existing medical imaging systems.

8. The vascular access guidance platform of claim 7, wherein the pre-procedural imaging data is obtained from medical imaging modalities selected from the group consisting of fluoroscopy systems, X-ray systems, and CT imaging systems.

9. The vascular access guidance platform of claim 1, wherein the processing system includes multi-modal image fusion processing configured to combine data from real-time sensors, pre-procedural imaging systems, and intraprocedural ultrasound through algorithms that create unified anatomical representations.

10. The vascular access guidance platform of claim 1, wherein the registration and tracking system is configured to utilize registration techniques selected from the group consisting of optical marker registration, spatial mapping using SLAM algorithms, bony landmark registration, and combinations thereof.

11. The vascular access guidance platform of claim 1, wherein the registration and tracking system includes fiducial bands equipped with registration sensors positioned over anatomical landmarks, the fiducial bands including multiple registration targets arranged in arc configurations that represent cross-sections of target anatomical regions.

12. The vascular access guidance platform of claim 1, wherein the registration and tracking system includes spatial mapping sensors using SLAM algorithms configured to process environmental scanning data to create comprehensive three-dimensional coordinate maps of patient anatomy.

13. The vascular access guidance platform of claim 1, wherein the system is configured to track medical instruments equipped with sensors within the extended reality visualization system, enabling registration of device position relative to patient anatomy in three-dimensional holographic space.

14. A method for providing vascular access guidance during medical procedures, comprising:
positioning registration sensors at predetermined anatomical locations on a patient to establish spatial reference points for augmented reality guidance during vascular access procedures;
capturing anatomical imaging data using multiple imaging sensor modalities configured to detect vascular structures and anatomical features;
generating an augmented representation of patient anatomy at the predetermined anatomical location based on the captured imaging data, wherein the augmented representation comprises a three-dimensional depiction of anatomy;
aligning the augmented representation with physical anatomy of the patient using spatial registration techniques that process sensor data from the registration sensors; and
displaying the aligned augmented representation through an extended reality visualization system, wherein the extended reality visualization system projects three-dimensional holograms overlaid directly onto patient anatomy.

15. The method of claim 14, wherein positioning registration sensors comprises identifying bony landmarks through palpation techniques and placing the registration sensors over the identified anatomical landmarks, wherein the registration sensors are selected from the group consisting of optical sensors configured to detect reflective passive markers or active LED markers, electromagnetic sensors for wireless tracking, acoustic sensors for position detection, and inertial measurement unit sensors for motion tracking.

16. The method of claim 15, wherein for arm registration procedures, the predetermined anatomical locations include wrist landmarks comprising a dorsal tubercle of radius, styloid process of ulna, and styloid process of radius, and elbow landmarks comprising a lateral supracondylar ridge, lateral epicondyle, and radial fossa.

17. The method of claim 14, wherein capturing anatomical imaging data comprises utilizing infrared cameras to detect subcutaneous vascular structures, visible light cameras to capture surface anatomy, depth-sensing technology to create three-dimensional surface maps, and ultrasound sensors to provide deeper tissue visualization.

18. The method of claim 14, wherein aligning the augmented representation comprises utilizing registration techniques selected from the group consisting of optical marker registration, spatial mapping using SLAM algorithms, bony landmark registration, and combinations thereof to maintain accurate spatial alignment throughout the procedure.

19. The method of claim 14, further comprising processing data from multiple sensor types simultaneously through coordinate transformation algorithms that align different sensor coordinate systems into a unified spatial reference frame, wherein the processing includes coordinate system calibration, temporal synchronization, and sensor fusion algorithms.

20. The method of claim 14, wherein displaying the aligned augmented representation comprises creating layered visualizations where pre-procedural imaging data provides baseline anatomical mapping, real-time sensor data provides current procedural context, and intraprocedural ultrasound data provides enhanced depth and tissue differentiation.
